(19) United States
(12) Patent Application Publication
(10) Pub. No.: US 2022/0371915 A1
(43) Pub. Date: Nov. 24, 2022

(54) System for Sanitizing Water in a Hot Tub

(71) Applicant: Hall Labs LLC, Provo, UT (US)

(72) Inventors: Michael Hall, Provo, UT (US); David R. Hall, Provo, UT (US); Prabhakar Ramaraj, Provo, UT (US)

(73) Assignee: Hall Labs LLC, Provo, UT (US)

(21) Appl. No.: 17/747,456

(22) Filed: May 18, 2022

Related U.S. Application Data
(60) Provisional application No. 63/190,040, filed on May 18, 2021.

(51) Int. Cl. C02F 1/02; A61L 2/26; A61L 2/04

(52) U.S. Cl. CPC C02F 1/02; A61L 2/26; A61L 2/04; C02F 2303/04; C02F 2103/42

(57) ABSTRACT

A system for sanitizing water in a hot tub is described. The system includes a sanitizing chamber, separate from the hot tub, and sized to hold a small portion of the water from the hot tub. A first conduit for conveys water from the hot tub to the sanitizing chamber. A second conduit conveys water from the sanitizing chamber to the hot tub. A pump is provided that moves water from the hot tub, through the first conduit into the sanitizing chamber and from the sanitizing chamber, through the second conduit, back into the hot tub. A heater heats the water in the sanitizing chamber to a temperature and for a time sufficient to destroy or deactivate undesirable microorganisms.

### CROSS-REFERENCE TO RELATED APPLICATIONS

This application claims priority to U.S. Provisional Patent Application No. 63/190,040, filed May 18, 2021 and titled “System for Sanitizing Water in a Hot Tub,” the entire disclosure of which is incorporated herein by reference.

### TECHNICAL FIELD

The invention relates to systems for cleaning and sanitizing water in a Hot Tub.

### BACKGROUND

A hot tub is a large tub filled with water, typically used for relaxation and/or hydrotherapy. Some hot tubs have jets for massage purposes. Hot tubs are sometimes referred to as spas or by the trade name Jacuzzi®. Unlike typical bathtubs, hot tubs are designed to be used by more than one person at a time, with most hot tubs accommodating four or more people.

Also, unlike typical bathtubs, most hot tubs are not drained and refilled after each use. Consequently, it is important to treat the water to keep it clear, odor-free and safe. To prevent skin irritation or worse, the water must also be pH balanced, that is, not too alkaline or acidic. It also needs to be sanitized to stay free of disagreeable and unhealthy microorganisms. Because of the high-water temperatures, hot tubs can pose particular health risks if not properly sanitized. For example, outbreaks of Legionnaires' Disease have been traced to poorly sanitized hot tubs. In conventional hot tubs, chlorine and/or bromine have been used as sanitizers. Saltwater chlorination is also used. Sanitation can also be aided by a non-chemical ozonator, although these are not typically used as primary sanitizers.

While conventional sanitizing systems are generally effective, the harsh chemicals can have deleterious effects on the users' skin. They can also have corrosive effects on the hot tub and its equipment, i.e. pumps, filters and heaters.

### SUMMARY

In a first aspect, the disclosure provides a system for sanitizing water in a hot tub. The system includes a sanitizing chamber, separate from the hot tub, and sized to hold a small portion of the water from the hot tub. A first conduit conveys water from the hot tub to the sanitizing chamber. A second conduit conveys water from the sanitizing chamber to the hot tub. A pump is provided that moves water from the hot tub, through the first conduit into the sanitizing chamber and from the sanitizing chamber, through the second conduit, back into the hot tub. A heater heats the water in the sanitizing chamber to a temperature and for a time sufficient to destroy or deactivate undesirable microorganisms.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a view of a hot tub with a sanitization system.

FIG. 2 is a graph of various sanitization cycles.

FIG. 3 is an exploded view of a heat exchanger.

FIG. 4 is a view of six heat exchangers in series between a hot tub and a sanitizing chamber.

FIG. 5 is a graph of temperature differences between heat exchangers when used in series.

FIG. 6 is a cut away view of an after-market sanitizing unit to be used with an existing hot tub.

FIG. 7 is a perspective view of an after-market sanitizing unit to be used with an existing hot tub.

FIG. 8 is a perspective view of an embodiment of the system, with the cover and part of the insulation removed to show the internal components.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, the term “undesirable microorganism” is intended to have a relatively broad meaning, referring both to pathogens, namely viruses, bacteria, fungi, protozoa and worms that can cause disease, as well as microorganisms that have other detrimental effects on the quality of the water, namely make the water odorous or unclear.

As used herein, the phrase “destroy or deactivate undesirable microorganisms” is intended to have a relatively broad meaning, not requiring complete elimination of undesirable microorganisms, but reducing levels enough to make a positive impact on the water quality in the hot tub. Naturally, total or near total elimination is most preferred.

As used herein, the phrase “small portion,” as in a “a small portion of the water from the hot tub” is used to express that only a minor portion of the water from the hot tub is held by the sanitizing chamber at a time. Preferably, this amount is less than 20 percent of the water, more preferably less than 10 percent, even more preferably less than 5 percent and most preferably less than 1 percent.

As noted above, the water in a hot tub can provide a haven for many undesirable microorganisms, such as odor causing bacteria and even disease-causing pathogens. Some of the undesirable microorganisms that can grow in water include Legionella, Pseudomonas, Cholera and Cryptosporidium. These microorganisms can cause rashes and illness. They are introduced to the hot tub in a variety of ways. Some are airborne and begin colonizing when the pathogen falls in the water and others are found on human skin and brought into the hot tub when a person enters the hot tub.

Heat can be an effective method for destroying or deactivating undesirable microorganisms in liquids. Boiling water has been used for centuries to purify water and make it safe to drink. Pasteurization is another method of sanitizing using heat. Pasteurization typically involves heating liquids to below 212° F. (100° C.) and holding them at that temperature for a time sufficient to destroy or deactivate those undesirable microorganisms.

The present invention uses heat to destroy or deactivate undesirable microorganisms, it is different from pasteurization and its use in other liquids. In pasteurization the liquid is heated, either all at once in a vessel, or as it all passes through a conduit. As such, pasteurization is a typically a one-time event. In contrast, the sanitizing system described herein, small volumes of the liquid are sanitized and then returned to the volume of the hot tub. Preferably, this is done as an ongoing process and is not a one-time event.

Hot tubs are known as good environments for many undesirable microorganisms. There are several reasons why hot tubs are good environments for microorganisms. These reasons include; having a large supply of water; being open to the air, at least sometimes; and being kept at temperatures where many undesirable microorganisms thrive.

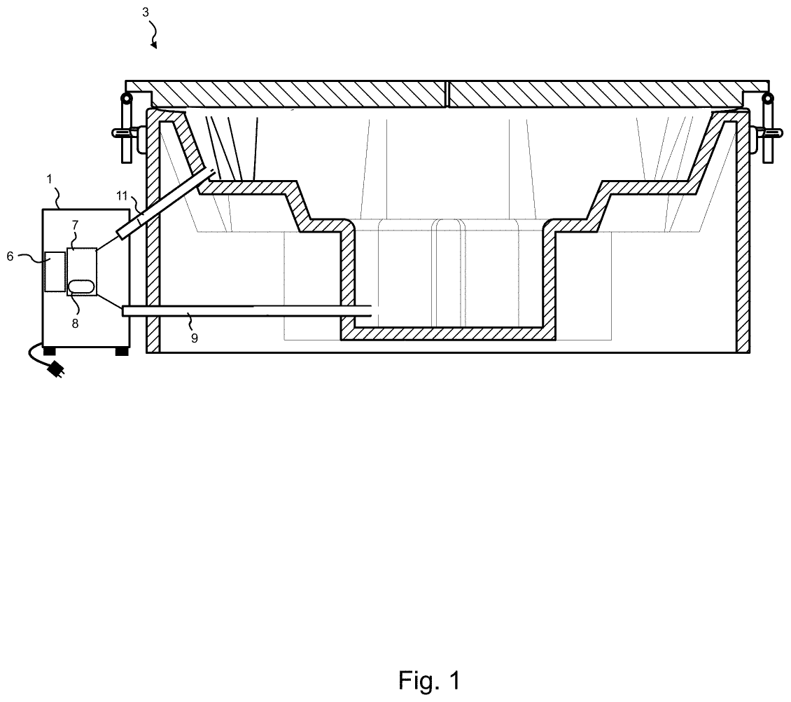

Now referring to FIG. 1, which shows an embodiment of the sanitization system in use with a hot tub. The system continually sanitizes small volumes of the water from the hot tub until all or substantially all of the water has been cycled through the sanitizing system. Also, after conventional pasteurization, the liquid is sealed, so as to prevent re-contamination and often refrigerated. In contrast, the hot tub described herein remains accessible to microorganisms in the environment and therefore recontamination. This recontamination or accessibility by contaminating microorganisms can be for any of a variety of reasons including, the hot tub is often open to allow people to get in and out of the hot tub, which leaves the surface of the water exposed to the environment. Additionally, the people entering the hot tub bring microorganisms into the hot tub with them. Some microorganisms are naturally found on the skin, while others may be present on the skin due to inadequate rinsing or cleaning before entering a hot tub. As such, the continuous operation of the system is even more advantageous. The sanitizing system 1 includes a sanitizing chamber 7, a heating element 8, and a pump 6 to move water from the hot tub to the sanitizing chamber and back to the hot tub. Within the sanitizing chamber 7, the water is heated and held at a temperature sufficiently high to destroy or deactivate the undesirable microorganisms.

The sanitizing chamber 7 needs to be large enough to heat a sufficient volume of water to cycle through the volume of water in the hot tub quickly enough to prevent growth of the undesirable microorganisms, while still being small enough to efficiently heat the water in the chamber. A smaller chamber will heat the water more quickly but will take longer to cycle through the entire volume of water in the hot tub. Conversely, a larger chamber will take longer to heat the water in the chamber but will cycle through the full volume of water in the hot tub more quickly. In some embodiments, the volume of the sanitizing chamber is between 0.5 liter and 5 liters. In the more preferred embodiment, the chamber is between 0.8 liter and 3 liters. In yet a more preferred embodiment, the chamber is between 1 liter and 2 liters. Stated another way, the small portion of water in the sanitizing chamber is preferably less than 20 percent of the water, more preferably less than 10 percent, even more preferably less than 5 percent and most preferably less than 1 percent.

The sanitizing system includes a heater 8 for heating the water in the sanitizing chamber 7. The heater may be any of a variety of heating methods. In some embodiments, the heating method is a flame-based element such as those found in many water heaters. In other embodiments, the heating method is a resistive heating element. In a yet other embodiments, the heating method is a waterproof resistive element. In another embodiments, the heater is a solar heater. Solar heaters are often used for heating water used for personal hygiene such as bathing and showering. While often used for heating bath water, solar heaters are capable of producing temperatures high enough to destroy or deactivate unwanted microorganisms, and so could be effectively used to heat and sanitize the water in the hot tub. There are several solar water heaters available commercially, such as those produced by Duda.

In some embodiments, the components of the sanitizing system are built into the hot tub. In such an embodiment, the components are built as a part of the hot tub at its construction, and do not need to be added onto the hot tub. In other embodiments, the components of the sanitizing system are retrofittable to a hot tub and can be added to an existing hot tub. Such an embodiment enables an existing and in use hot tub to have the components of the sanitizing system attached and incorporated into the hot tub.

The sanitizing chamber needs to be constructed of a material that will hold water and stand up to high temperatures. The type of heating element used for heating the water also influences the material the sanitizing chamber is constructed of. When the heating method is a flame-based element, the sanitizing chamber needs to be constructed of a material that will be able to have a flame heat the chamber and will transfer the heat to the water while not destroying the chamber. Such materials include metals, like copper, steel, stainless steel, aluminum, alloys or combinations of each of these and other metals, as well as some ceramic materials. There are several issues with using a flame-based heating element including needing an attachment for the flammable gas, having the flammable gas available in the location the hot tub is positioned, and the safety of the gas itself. In the preferred embodiment, the heating method is a resistive heating element. Utilizing a resistive heating element enables the chamber to be made of a wide variety of materials, including metals, such as copper, steel, stainless steel, aluminum, alloys or combinations of each of these and other metals, ceramics, and polymers, such as PVC, ABS, and carbon fiber. In one embodiment, the heating method is a waterproof resistive heating element. Utilizing a waterproof resistive heating element further expands the possibilities for the location of the heating method as the waterproof resistive heating element can be placed inside the sanitizing chamber.

In one embodiment, a first conduit 9 conveys water from the hot tub to the sanitizing chamber, and a second conduit 11 conveys water from the sanitizing chamber to the hot tub. When the sanitized water is placed back in the hot tub it is possible that the full volume of the water in the hot tub will not have made it through the sanitization chamber. Sanitizing the majority of the water in the hot tub will result in greatly reduced microbial load. However, there is a possibility of a small volume of water, and thus anything in the water, not being processed through the sanitization chamber.

The conduits are composed of materials designed for transporting water, these materials include metal tubing such as copper, stainless steel, aluminum, polymers such as PVC, and rubber. Each of these materials have benefits and drawbacks. In the preferred embodiment, the conduits are made of a polymer. Polymers are advantageous because they are generally good insulators. By using a polymer, less heat is lost during the time the water is transported from the sanitizing chamber to the hot tub. Polymer conduits are most advantageous in embodiments utilizing heat exchangers, as will be explained later. In some embodiments, heat loss during transport time is not a detriment, in such instances, conduit made from a metal would be more advantageous. The metal conduit could be used specifically to allow heat to escape from the water before reentering the hot tub. Metal conduits are more advantageous in embodiments where the temperature difference between the water temperature in the hot tub and the water temperature in the sanitizing chamber are high, and there are no heat exchangers in the system. Metal conduit may also be used in embodiments with heat exchangers, provided the conduits were wrapped in an insulative material.

There are various temperature schedules for dealing with undesirable microorganisms in water. For example, OSHA recommends raising the temperature of a water heater to 158° F. for 24 hours to destroy or deactivate legionella. In a paper published in Applied and Environmental Microbiology, for pasteurization of drinking water in developing countries, the researchers stated that a temperature of 149° F. for 6 minutes is enough to destroy or deactivate all germs, viruses, and protozoa. “Ciochetti, D. A., and Metcalf, R. H., Pasteurization of Naturally Contaminated Water with Solar Energy, Applied and Environmental Microbiology, 47:223-228, 1984.”

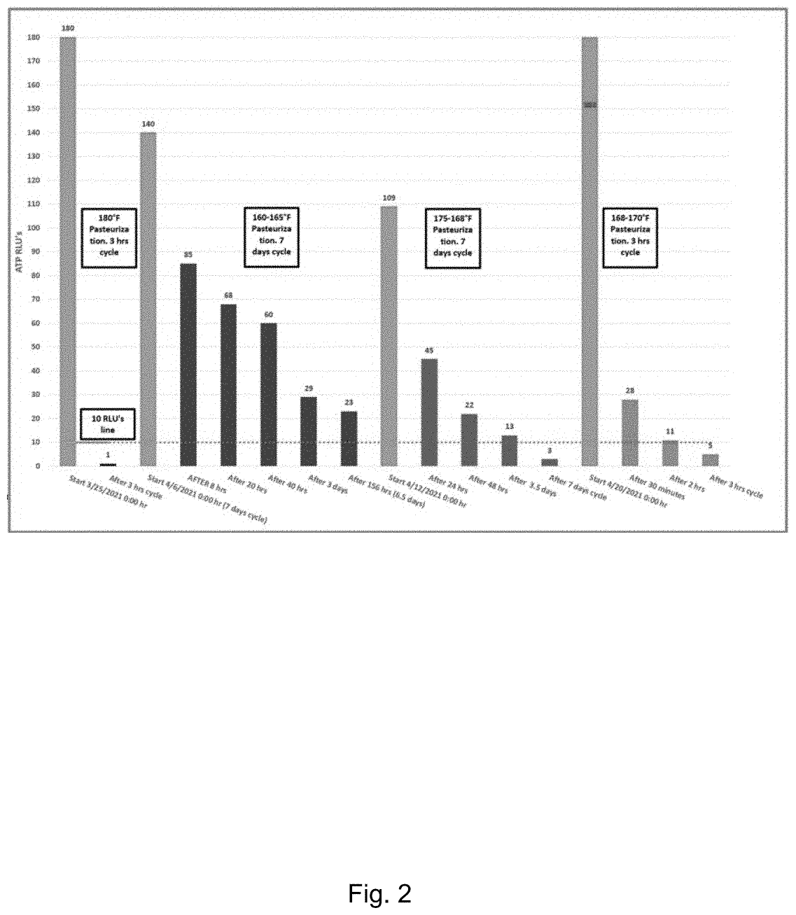

Various temperature and cycle times can be used. FIG. 2 is a graph showing contamination before and after various sanitization cycles in the water of a hot tub. Water from the hot tub is conducted from the hot tub, through the first conduit to the sanitizing chamber. In the sanitizing chamber the water is brought to the desired temperature and held at that temperature for a period long enough to destroy or deactivate any undesirable microorganisms. Temperatures in the sanitizing chamber range from 160-180° F. and hold times range from 10 seconds to one minute. In some embodiments the hold time is between 15 and 45 seconds. In more preferred embodiments, the hold time is between 22 and 38 seconds. In the most preferred embodiment, the hold time is 30 seconds. In some embodiments, the temperature is between 160-180° F. In other embodiments, the temperature is between 164-176° F. In the most preferred embodiment, the temperature is above 168° F. and below 172° F.

In some embodiments, the volume of the water in the hot tub is cycled through the sanitizing chamber over a period between 1 hour and 10 days. In a more preferred embodiment the cycle occurs over a period between 3 and 7 days. In a second more preferred embodiment, the cycle occurs over a 3-hour period. The most preferred method to sanitize the water is to heat the water to between 168-172° F., hold for 30 seconds, and cycle through all the water in the hot tub in 3 hours. The water in the hot tub can be kept sanitized by running cycles through the sanitizing process of the sanitizing chamber. The frequency of the cycles is dependent on use and cleanliness of the water entering the hot tub and the cleanliness of the environment around the hot tub.

Typically, water in a hot tub is kept at between 100° F. and 102° F. The sanitizer is optimally configured to heat the water to 168-170° F. which leads to a temperature difference of 68° F. to 70° F. Heating the water by 68° F. to 70° F. in the sanitizing chamber is inefficient, both in terms of energy input and the time it takes to reach the desired temperature. To raise the temperature of one gallon of water 1° F. requires 8.33 BTUs. Raising 1 gallon of water 70° F. would therefore require 583 BTUs. A hot tub holding 500 gallons of water would consume 291,500 BTUs. Additionally, putting the water heated to 170° F. directly back into the hot tub can be dangerous. If someone is sitting near the inlet from the sanitization chamber, they could be injured by the incoming heated water.

Utilizing heat exchangers as part of the first and second conduits solves both of these problems. A heat exchanger is a sealed chamber with two sides divided by a thermal conductive plate. There are several options for commercially available heat exchangers, the inventors chose a commercially available brazed plate heat exchanger. A heat exchanger exchanges heat between the water traveling, in the first conduit, from the hot tub to the sanitizing chamber and the water traveling, in the second conduit, from the sanitizing chamber to the hot tub. Water from the hot tub enters one side of the heat exchanger. The water from hot tub is held in the hot tub water side, or first conduit side, of the heat exchanger while water from the sanitizing chamber enters the sanitizing chamber side, or second conduit side, of the heat exchanger. The water from the hot tub and the sanitizing chamber are held in the heat exchanger for a dwell time. During the dwell time the heat from the water on sanitizing chamber side of the exchanger passes through a thermal conductive plate to the water on the hot tub side of the heat exchanger. The water coming from the hot tub is heated. When that water moves to the sanitizing chamber, less energy is used in the sanitizing chamber to bring it up to the appropriate temperature. Simultaneously, the water moving from the sanitizing chamber is cooled and is less dangerous as it moves into the hot tub.

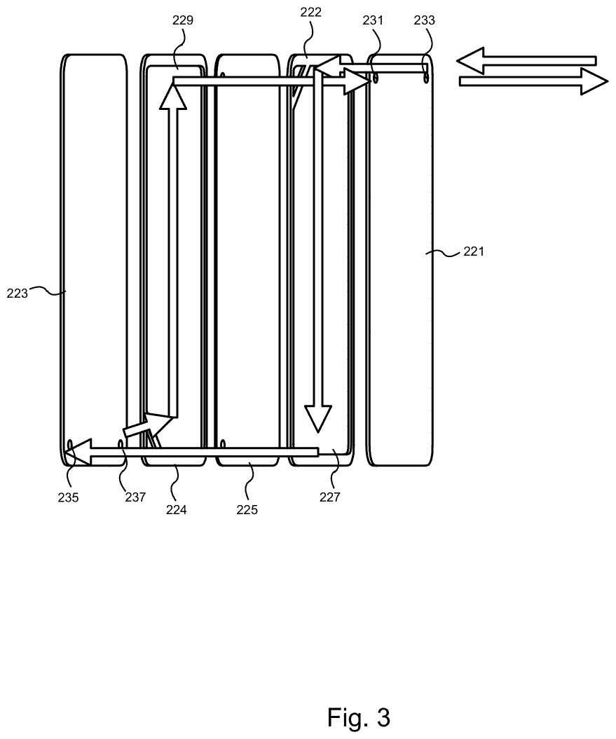

One embodiment, of a heat exchanger such as that depicted in FIG. 3. The heat exchanger has a first cover plate 221, a second cover plate 223, a thermal conductive plate 225, a first gasket 222 and a second gasket 224. In the space created by the first cover plate 221, the first gasket 222, and the thermal conductive plate 225 is a first water storage chamber 227. In the space created by the second cover plate 223, the second gasket 224, and the thermal conductive plate 225 is a second water storage chamber 229. The first cover plate includes an inlet 233 and an outlet 231. The second cover plate 223 includes an inlet 237 and an outlet 235. Water from the hot tub is cycled into the first water storage chamber 227 by passing through the inlet 233 from the hot tub. Simultaneously, water from the sanitizing chamber is cycled into the second water storage chamber 229 through the inlet 237 from the sanitization chamber. The water coming from the sanitization chamber is hotter than the water coming from the hot tub. Heat from the water coming from the sanitization chamber is held in the second water storage chamber 229 and heats the thermal conductive plate 225. The thermal conductive plate 225 then heats the water in the first water storage chamber 227 which was moved from the hot tub. The water is held in the water storage chambers for a set time. This time is generally referred to as a dwell time. Following the dwell time, the water in the first water storage chamber 227 is moved out of the heat exchanger through outlet 235, and the water in the second water storage chamber 229 is moved out of the heat exchanger through outlet 231.

The use of previously heated water to raise the temperature of the water on the way to the sanitizing chamber along with the resultant smaller temperature difference provided by utilizing heat exchangers is helpful for conserving energy. The energy to heat up the water on its way to the sanitizing chamber could have been dissipated to the environment as heat, instead it is used to bring the water from the hot tub to a higher temperature so less energy is needed to bring the water up to the full sanitizing temperature in the sanitizing chamber. Each heat exchanger holds a smaller volume than the volume of the sanitizing chamber, because of this some of the volume of the water will remain in the sanitizing chamber longer than the hold time for destroying and deactivating unwanted microorganisms. In one embodiment, the pump is programmed to cycle on to move the water through the conduits to each heat exchanger and cycle off to leave the water in the heat exchangers for a programmed dwell time. Different dwell times will result in different temperature changes. The number of heat exchangers also affects the change in temperature.

In one embodiment, a multi-stage heat exchanger is made by stacking multiple chambers one against the next, so that as the warm water flows from the first chamber to the last it is getting cooled in each stage. Likewise, as the cool water flows in the opposite direction from its first chamber to the last, it is progressively getting heated. The number of heat exchanging chambers in the stack can be increased to increase the efficiency of the heat recovery provided, with the goal of decreasing the difference in the temperature at the inlet and outlet of the stack. Insulation between the heat exchanging chambers in otherwise adjacent stages is important so that heat is not allowed to travel outside the chambers. While a single heat exchanger does exchange the heat between the two sides of the water, the addition of multiple heat exchangers more effectively cools the water on its way to the hot tub and more effectively heats the water on its way to the sanitizing chamber.

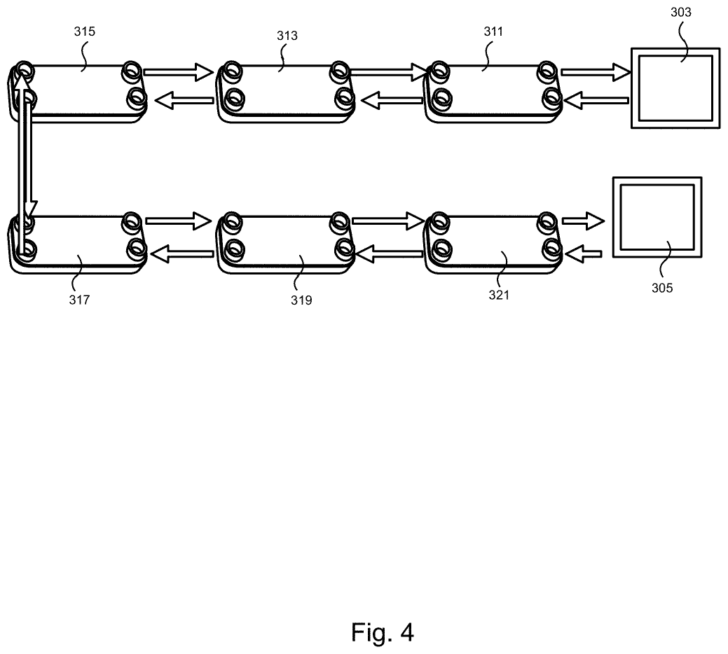

FIG. 4 shows six separate heat exchangers lined up in series to heat the water on its way to the sanitizing chamber and simultaneously to cool the water on its way to the hot tub. In the embodiment depicted, the water begins at the hot tub 303. As the water from the hot tub is moved to the first heat exchanger 311. Water from the sanitizing chamber 305 has already passed through five heat exchangers 313, 315, 317, 319, and 321 and transferred much of the heat originally in the water as it left the sanitizing chamber to the water moving from the hot tub 303 to the sanitizing chamber 305. Throughout the series of heat exchangers, the temperature of the water moving from the hot tub 303 to the sanitizing chamber 305 gets hotter the closer to the sanitizing chamber 303 it travels. Simultaneously, the temperature of the water moving from the sanitizing chamber 305 to the hot tub 303 gets colder. Each heat exchanger transfers some of the heat to the water that is moving from the hot tub to the sanitizing chamber 305. In one example, the water in the hot tub begins at 101.8° F. The water from the hot tub enters the first heat exchanger 311 at 101.8° F. In the first heat exchanger the temperature is raised, the average temperature of the water is 103.8° F., this is the average of the temperature of the water coming from the hot tub 303 and the water coming from the sanitization chamber 305. The water from the hot tub is heated by water from the sanitization chamber. The water then moves from the first heat exchanger 311 to the second heat exchanger 313. The average temperature of the water in the second heat exchanger is 116° F. The average temperature of the water in the third heat exchanger 315 is 128° F. The average temperature of the water in the fourth heat exchanger 317 is approximately 140° F. The average temperature of the water in the fifth heat exchanger 319 is 152° F. The average temperature of the water in the sixth heat exchanger 321 is 164° F. The temperature in the sanitization chamber 305 is 170° F. Due to the heat exchangers the water that enters the sanitization chamber only needs to be raised about 6° F. to reach the final sanitization temperature. This means that each gallon of water will require approximately 50 BTUs to reach sanitization temperature. A 500-gallon hot tub would thus require 25,000 BTUs to go through the sanitization process. Without the heat exchangers, the water would need to be heated by about 70° F. using approximately 291,500 BTUs. This is an energy savings of more than tenfold.

The pump used to convey water from the hot tub to the sanitizing chamber and from the sanitizing chamber to the hot tub is programmed to leave the water in the heat exchangers for a set dwell time. It does this by cycling on to pump the water through the conduit to each heat exchanger and the sanitizing chamber, and to cycle off leaving the water in the heat exchangers for a dwell time. In one embodiment, the sanitization system, with six heat exchangers is used. In one embodiment, the dwell time is between 15 and 45 seconds. In a more preferred embodiment, the dwell time is between 22 and 38 seconds. In the most preferred embodiment, the dwell time is 30 seconds. Different dwell times will result in different temperature changes. The number of heat exchangers also affects the change in temperature.

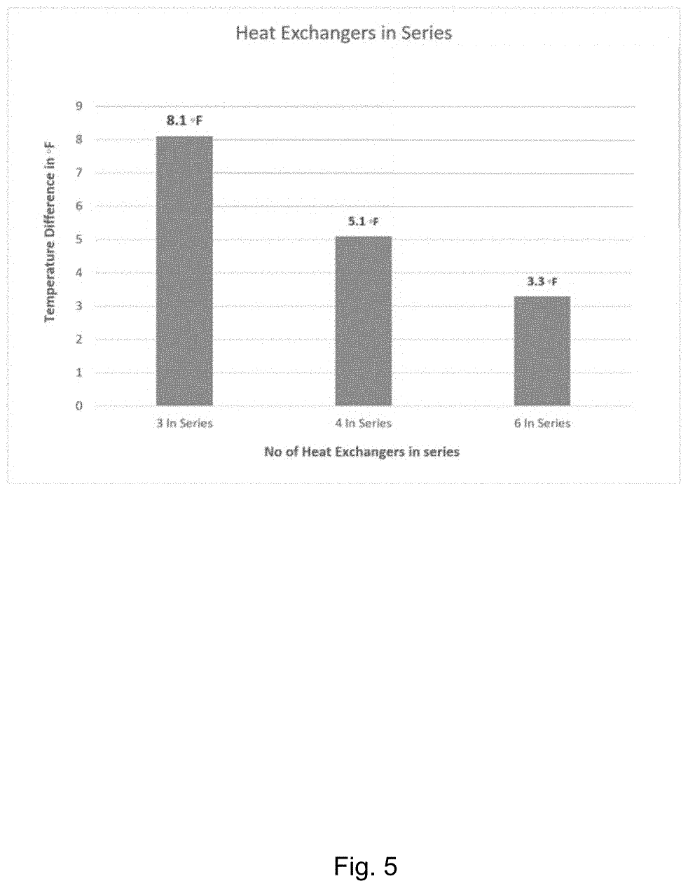

FIG. 5 is a graph depicting the effect of the number of heat exchangers in a sanitization system used with a hot tub. In this example, the flow rate of the pump was set to 320 ml/min, with the pump on for 30 seconds and the pump off for 30 seconds. In one embodiment, three heat exchangers are used with a 30 second dwell time, this results in a temperature difference between the temperature of the water as it exits the hot tub and the temperature of the water as it reenters the hot tub of 8.1° F. In a second embodiment, the number of heat exchangers is four and the temperature difference between the temperature of the water as it exits the hot tub and the temperature of the water as it reenters the hot tub of 5.1° F. In a more preferred embodiment, six heat exchangers and a 30 second dwell time are used, and the difference in temperature between the temperature of the water as it exits the hot tub and the temperature as it reentered the hot tub is 3.3° F. The embodiments here described heat exchangers lined up in a series. One limiting factor to the number of heat exchangers is space. In other words, the heat exchangers need to fit within the space available. With larger space, or with smaller heat exchangers, more heat exchangers can be used. Adding more heat exchangers increases efficiency, because the temperature change between each subsequent heat exchanger decreases.

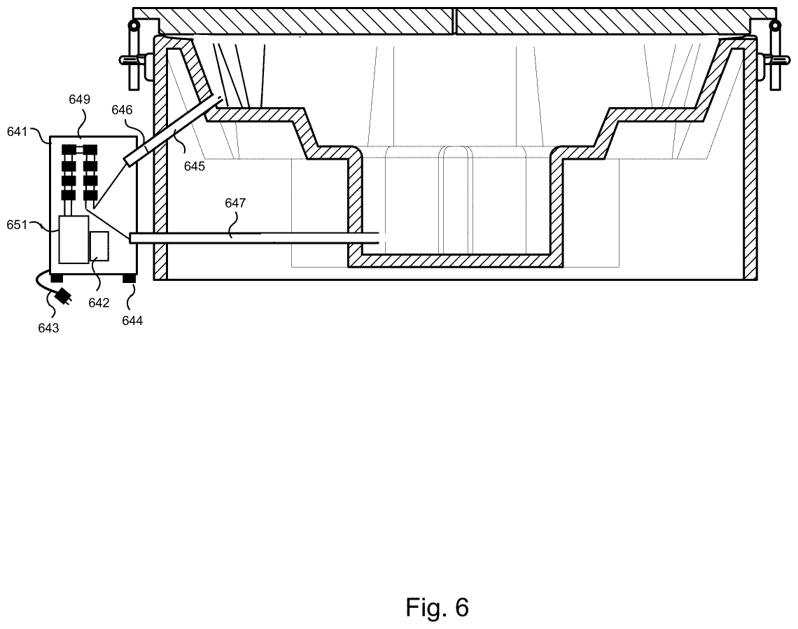

FIG. 6 depicts an internal view of one embodiment which is adapted to be added as an aftermarket device to an existing hot tub. The unit includes a housing 641, with vibration isolating feet 644. The unit is powered by a cord 643. For safety, it is preferably to include a Ground Fault Current Interrupter (GFCI) device in the cord. Water is brought from the hot tub to the unit by pump 642 through water line 645. Preferably, water line 645 includes a filter 646, such as a simple screen, to prevent solids from entering the unit. Alternatively, a more complex filter, such as a replaceable, pleated cartridge can be put in the line. Water passes through water intake line 645 and passes through a series of heat exchangers 649 on its way to the sanitization chamber 651. Sanitized water is returned to the hot tub through water line 647.

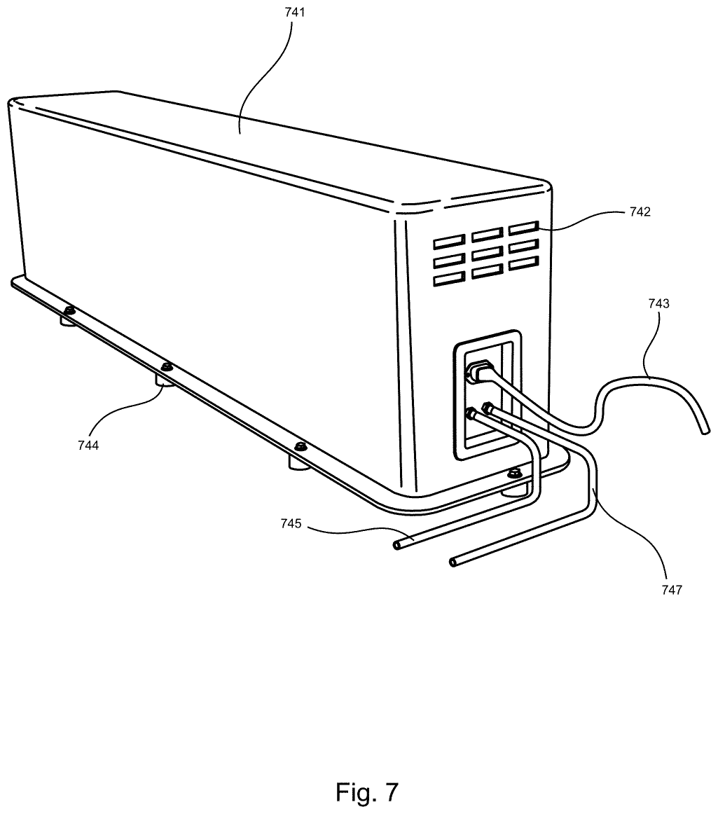

FIG. 7 depicts an outside view of an embodiment of the aftermarket device for use with an existing hot tub. The unit includes a housing 741, with air vents 742 and vibration isolating feet 744. The unit is powered by a cord 743. For safety, it is preferably to include a Ground Fault Current Interrupter (GFCI) device in the cord. Water is brought from the hot tub to the unit through water line 745. Preferably, water line 745 includes a filter, such as a simple screen, to prevent solids from entering the unit. Alternatively, a more complex filter, such as a replaceable, pleated cartridge can be put in the line. Water passes through water intake line 745 and passes through a series of heat exchangers 749 on its way to the sanitization chamber 751. Sanitized water is returned to the hot tub through water line 747.

In other embodiments, the system incorporates or is used with other water filtering or treatment technology, such as ion exchange or electronic descaling to remove minerals.

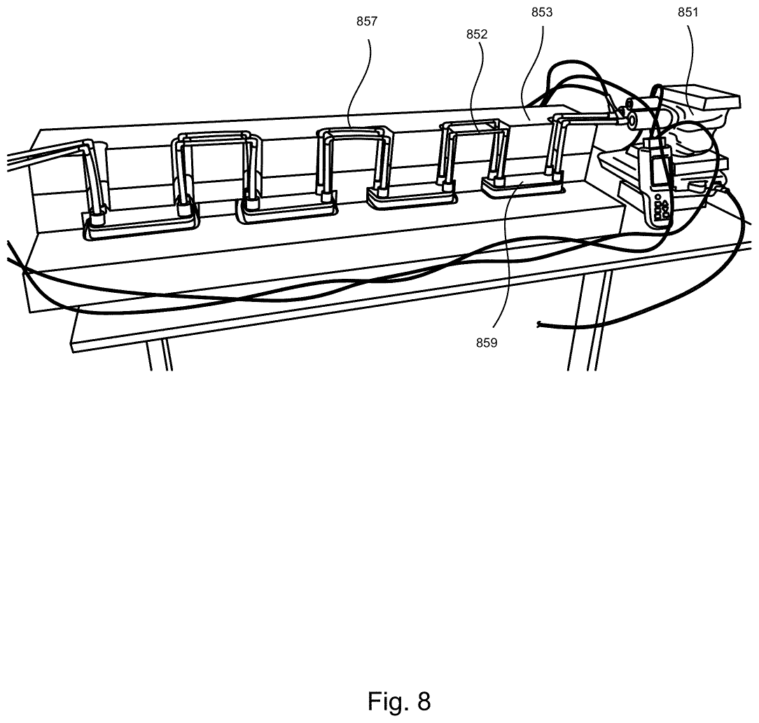

FIG. 8 shows an embodiment of a unit with the housing and some of the insulation removed to show the internal parts. This unit includes a pump and heater 851. A series of four heat exchangers 859 are placed in series and fed water from the hot tub through water lines 855. The water is returned to the hot tub through water lines 857. To avoid heat loss to the environment, the pump, heater, sanitizing chamber, heat exchangers and water lines are all surrounded by a heat insulating material 853, such as polystyrene foam. The wires and gauges shown in FIG. 9 are there for testing.

In the preferred embodiment, the unit is sized and shaped to fit under an existing step for accessing the hot tub. Alternatively, it can be manufactured as a step itself. Still alternatively, it can be manufactured into another hot tub accessory, such as an insulative cover or a drinks and towel stand.

The invention has been described with reference to various specific and preferred embodiments and techniques. Nevertheless, it is understood that many variations and modifications may be made while remaining within the spirit and scope of the invention.

All patents and published patent applications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A system for sanitizing water in a hot tub comprising:
a sanitizing chamber, separate from the hot tub, and sized to hold a small portion of the water from the hot tub;
a first conduit for conveying water from the hot tub to the sanitizing chamber;
a second conduit for conveying water from the sanitizing chamber to the hot tub;
a pump configured to move water from the hot tub, through the first conduit, into the sanitizing chamber and from the sanitizing chamber, through the second conduit, into the hot tub;
a heater configured to heat water in the sanitizing chamber to a temperature and for a time sufficient to destroy or deactivate undesirable microorganisms.

2. The system of claim 1, wherein the temperature in the sanitizing chamber is above 168° F.

3. The system of claim 1, wherein the volume of the sanitizing chamber and the rate of the pump is selected to achieve the time sufficient to destroy or deactivate undesirable microorganisms.

4. The system of claim 3, wherein the water is held in in the sanitizing chamber for at least 30 seconds.

5. The system of claim 1, wherein the pump is programed to cycle on and off so that water is held in the sanitizing chamber for the time sufficient to destroy or deactivate undesirable microorganisms.

6. The system of claim 5, wherein the pump is cycled off for between 15 and 45 seconds to achieve the time sufficient to destroy or deactivate undesirable microorganisms.

7. The system of claim 6, wherein the pump is cycled off for 30 seconds.

8. The system of claim 1, wherein the first and second conduit each comprise at least one heat exchanger, whereby water in the first conduit is heated by the water in the second conduit and water in the second conduit is heated by water in the first conduit.

9. The system of claim 8, wherein the first and second conduits each comprise at least three heat exchangers.

10. The system of claim 9, wherein heat recovery effected by the at least three heat exchangers allow water to be taken out of the hot tub, to be conveyed through the first conduit with heat exchangers, to dwell in the sanitizing chamber at a sanitizing temperature, to be conveyed through the second conduit with heat exchangers, and to be returned to the hot tub with an increased temperature of the water upon return to the hot tub of no more than 8.1° F.

11. The system of claim 8, wherein the first and second conduits each comprise at least six heat exchangers.

12. The system of claim 11, wherein heat recovery effected by the at least six heat exchangers allows water to be taken out of the hot tub, to be conveyed through the first conduit with heat exchangers, to dwell in the sanitizing chamber at a sanitizing temperature, to be conveyed through the second conduit with heat exchangers, and to be returned to the hot tub with an increased temperature of no more than 3.3° F.

13. The system of claim 12, wherein the pump is programed to cycle on and off so that water is held in the sanitizing chamber and each of the heat exchangers for a time between 15 and 45 seconds.

14. The system of claim 1, wherein the hot tub comprises a primary heater for heating the water in the hot tub, and wherein the heater heating water in the sanitizing chamber is independent of the primary heater.

15. The system of claim 14, wherein the heater heating water in the sanitizing chamber comprises a resistive heating element.

16. The system of claim 14, wherein the hot tub comprises a primary heater for heating the water in the hot tub, and wherein the heater capable of heating water in the sanitizing chamber shares a common heat source with the primary heater.

17. The system of claim 1, wherein the hot tub contains a volume of water and wherein the pump runs at a rate so that the volume pumped into the sanitizing chamber in a period between 3 and 7 days equals the volume of water in the hot tub.

18. The system of claim 1, wherein the hot tub contains a volume of water and wherein the pump runs at a rate so that the volume pumped into the sanitizing chamber in a period between 2 and 4 hours equals the volume of water in the hot tub.

19. The system of claim 1, wherein the components are adapted to be retrofitted on an existing hot tub.

20. The system of claim 1, wherein the components are built into the hot tub.
